(19) United States
(12) Patent Application Publication
(10) Pub. No.: US 2012/0329767 A1
(43) Pub. Date: Dec. 27, 2012

(54) Methods and Compositions for Treating Cancer

(76) Inventors: Michel Pfeffer, Strasbourg (FR); Christian Gaiddon, Strasbourg (FR); Claude Sirlin, Bischeim (FR); Jean-Paul Collin, Strasbourg (FR); Jean-Philippe Loeffler, Berstett (FR)

(21) Appl. No.: 13/381,276

(22) PCT Filed: Jun. 28, 2010
(86) PCT No.: PCT/FR10/51367

§ 371 Date: Jul. 26, 2012

(51) Int. Cl. A61K 31/555; A61P 35/00; A61P 35/02; C07F 15/00

(52) U.S. Cl. 514/185; 514/188; 546/4

(57) ABSTRACT

The present invention relates to uses or methods for treating proliferative diseases, in particular cancer, implementing ruthenium compounds, as well as to composition containing same.

The present invention relates to uses or methods for treating proliferative diseases, in particular cancer, using ruthenium compounds, and to compositions containing the same.

It is known that complexes of platinum have major anti-tumour activities. The most known of these is cisplatin, which is commonly used for the clinical treatment of numerous cancers. The resistance of some cancer cells and the intrinsic toxicity of platinum are one of problems encountered when using this compound. Since the 70s, research has been intensified to find molecules which could substitute for cisplatin, and for some years compounds containing ruthenium have appeared to be a possible alternative of interest to those containing platinum. Some complexes of ruthenium have therefore already been described as possible alternatives for anti-cancer treatment.

There is consequently a need for novel anti-cancer agents which could be an alternative to those currently used, which are advantageously more efficient and/or exhibit fewer adverse side effects.

The present invention therefore describes ruthenium compounds which display anti-tumour properties of particular interest. These compounds are organometallic compounds i.e. they contain at least one covalent Carbon-Metal bond (C-M, M being ruthenium). In addition, this C—Ru bond is stabilized by two other intramolecular nitrogen-ruthenium bonds (N—Ru), the nitrogen atoms being elements of the organic part linked to ruthenium via the carbon atom.

In this type of atom arrangement, ruthenium therefore belongs to a cyclic entity and this class of compounds is commonly called the class of cyclometallated compounds by chemists in this field. The cyclic entity containing ruthenium is called a metallocycle. In said metallocycle, ruthenium is therefore linked to an organic ligand both via a covalent carbon-ruthenium bond and via two bonds of donor-acceptor type (Lewis acid-base, or coordination bond) nitrogen-ruthenium. The existence of said metallocycle in an organometallic molecule imparts particular properties thereto in terms of reactivity and thermodynamic stability. Various types of carbon atoms (aromatic, benzyl or aliphatic) can be metallated and the type of bond between the donor atom (nitrogen) and the carbon atom can be modified in multiple ways.

According to a first aspect, the object of the present invention is a pharmaceutical composition which, in a pharmaceutically acceptable medium, comprises at least one complex ruthenium compound of the following general formula:

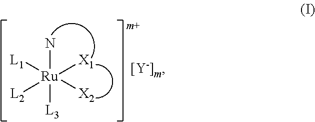

formula (I) in which:
L₁, L₂, L₃ the same or different each represent either a donor ligand of 2 electrons via a nitrogen, oxygen, phosphorus or sulfur atom, or a halogen atom, Y⁻ is a counter-ion (when m=1), m is 0 or 1, X₁ and X₂ differ from each other one representing a nitrogen atom and the other a carbon atom, between X₁ and X₂ as represented by a curved line there is a succession of atoms which, with X₁, X₂ and Ru represented in the formula, form a ring composed of 5 to 8 atoms, and between N and X₁ represented by the other curved line there is a succession of atoms which, with the nitrogen atom, X₁ and Ru represented in the formula, form a ring composed of 5 to 8 atoms.

Within the context of the invention, by the term <<pharmaceutically acceptable medium>> is meant substances such as excipients, vehicles, additives, buffers which are conventionally used in combination with the active ingredient(s) for the preparation of a medicinal product. The choice of said media essentially depends on the contemplated route of administration.

The compounds of the invention can be in the form of pharmaceutically acceptable salts, solvates and/or pro-drugs. Pro-drugs are variants of the compounds of the invention which can be converted in vivo to compounds of general formula according to the invention.

By <<halogen atom>> it is meant to designate a fluorine, chlorine, bromine or iodine atom. Advantageously the halogen atom is a chlorine atom.

The donor ligands of two electrons by a nitrogen, oxygen or sulfur atom include for example H₂0, di((C₁₋₆)alkyl)O, di((C₁₋₆)alkyl)S, di((C₁₋₆)alkyl)S(O), (C₁₋₆)alkylSO₃⁻, di((C₁₋₆)alkyl)C═O, (C₁₋₆)alkylCO₂⁻.

Other possible ligands for L₁, L₂ and/or L₃ particularly include nitrile ligands, such as ligands of formula (C₁₋₆)alkylCN (in particular CH₃CH) and pyridine ligands, optionally substituted, on one or more carbon atoms of the pyridine rings, by a halogen atom, a radical: alkyl, aryl, hydroxyl, alcoxyl (O-alkyl), aryloxyl (O-aryl), carboxylic acid, ester (CO₂-alkyl), thiol, thioether (S-alkyl), sulfinic acid, sulfonic acid, nitro, nitroxyl, amine (N(alkyl or aryl)ₓH₂₋ₓ where x is an integer possibly being 0, 1 or 2), trialkylammonium (N(alkyl or aryl)_{y}H_{3-y}⁺ where y is an integer possible being 0, 1, 2 or 3), hydroxylamine (N(OH)_{Z}(alkyl or aryl)_{2-z} where z is an integer possibly being 1 or 2), hydrazine, azo(N═N-(alkyl or aryl), diazonium, amide (CO—NH_{W}(alkyl or aryl)_{2-w} where w is an integer possibly being 0, 1 or 2) or cyano. The definitions of the chemical radicals given in this paragraph are valid for all the uses of these terms in the present application.

Among other ligands, particular mention may be made of the primary amines (C₁₋₆)alkylNH₂ such as methylamine or ethylamine.

The donor ligands of two electrons via a phosphorus atom include the ligands of phosphine type. Advantageously, they are of formula P(Ph)₃₋ₓ(alkyl)ₓ, where x represents 0, 1 or 2, preferably x represents 2, and Ph represents the phenyl group. Amongst these ligands, particular mention may be made of P(Ph)(CH₃)₂.

According to the invention, the term <<alkyl>> designates a straight or branched, saturated or unsaturated hydrocarbon radical advantageously having 1 to 6 carbon atoms, such as methyl, ethyl, propyl, isopropyl, butyl, isobutyl, tert-butyl, pentyl, neopentyl, n-hexyl, etc. The groups having 1 to 4 carbon atoms are preferred. The alkyl groups can be substituted by an aryl group in which case they are called arylalkyl groups. Examples of arylalkyl groups are notably benzyl and phenethyl. The alkyl or arylalkyl groups may optionally have one or more substituents, chosen in particular from among a halogen atom, a radical alkyl, aryl, hydroxyl, alcoxyl(O-alkyl), aryloxyl (O-aryl), carboxylic acid, ester (CO₂-alkyl), thiol, thioether (S-alkyl), sulfinic acid, sulfonic acid, nitro, nitroxyl, amine (N(alkyl or aryl)ₓH₂₋ₓ where x is an integer possibly being 0, 1 or 2), trialkylammonium (N(alkyl or aryl)_{y}H_{3-y}⁺ where y is an integer possibly being 0, 1, 2 or 3), hydroxylamine (N(OH)_{z}(alkyl or aryl)_{2-z} where z is an integer possibly being 1 or 2), hydrazine, azo (N═N-(alkyl or aryl)), diazonium, amide (CO—NH_{w}(alkyl or aryl)_{2-w} where w is an integer possibly being 0, 1 or 2) or cyano.

The <<aryl>> groups are mono-, bi- or tricyclic aromatic hydrocarbons, optionally interrupted by at least one heteroatom (in particular O, S or N). Preferably, the aryl groups include monocyclic or bicyclic aromatic hydrocarbon systems having 6 to 18 carbon atoms, further preferably 6 carbon atoms. Mention may be made for example of the phenyl, naphthyl and bi-phenyl groups. When they are interrupted by heteroatoms, the aryl groups include the pyridyl, imidazoyl, pyrrolyl and furanyl rings. The aryl groups may optionally have one or more substituents notably chosen from among a halogen atom, a radical alkyl, aryl, hydroxyl, alcoxyl (O-alkyl), aryloxyl (O-aryl) radical, carboxylic acid, ester (CO₂ alkyl), thiol, thioether (S-alkyl), sulfinic acid, sulfonic acid, nitro, nitroxyl, amine (N(alkyl or aryl)ₓH₂₋ₓ where x is an integer possibly being 0, 1 or 2), trialkylammonium (N(alkyl or aryl)_{y}H_{3-y}⁺ where y is an integer possibly being 0, 1, 2 or 3), hydroxylamine (N(OH)_{z}(alkyl or aryl)_{2-z} where z is an integer possibly being 1 or 2), hydrazine, azo (N═N(alkyl or aryl)), diazonium, amide (CO—NH_{w}(alkyl or aryl)_{2-w} where w is an integer possibly being 0, 1 or 2) or cyano.

According to one particular embodiment, two or three of the groups L₁, L₂ and L₃ may be linked together by at least one covalent bond. In this respect particular mention may be made of the bipyridine, phenanthroline or terpyridine units, optionally substituted, in particular by at least one halogen atom, a radical alkyl, aryl, hydroxyl, alcoxyl (O-alkyl), aryloxyl (O-aryl), carboxylic acid, ester (CO₂-alkyl), thiol, thioether (S-alkyl), sulfinic acid, sulfonic acid, nitro, nitroxyl, amine (N(alkyl or aryl)ₓH₂₋ₓ where x is an integer possibly being 0, 1 or 2), trialkylammonium (N(alkyl or aryl)_{y}H_{3-y}⁺ where y is an integer possibly being 0, 1, 2 or 3), hydroxylamine (N(OH)_{z}(alkyl or aryl)_{2-z} where z is an integer possibly being 1 or 2), hydrazine, azo (N═N-(alkyl or aryl)), diazonium, amide (CO—NH_{w}(alkyl or aryl)_{2-w} where w is an integer possibly being 0, 1 or 2) or cyano.

Preferably, the compounds of the invention have one, two or three L₁, L₂ and L₃ groups representing a donor ligand of two electrons via the nitrogen atom.

In particular, L₁, L₂ and/or L₃ may alone, or two by two, or in three represent a pyridine, a bipyridine, a phenanthroline or a terpyridine group, the said groups optionally being substituted, in particular by at least a halogen atom, a radical alkyl, aryl, hydroxyl, alcoxyl (O-alkyl), aryloxyl (O-aryl), carboxylic acid, ester (CO₂-alkyl), thiol, thioether (S-alkyl), sulfinic acid, sulfonic acid, nitro, nitroxyl, amine (N(alkyl or aryl)ₓH₂₋ₓ where x is an integer possibly being 0, 1 or 2), trialkylammonium (N(alkyl or aryl)_{y}H_{3-y}⁺ where y is an integer possibly being 0, 1, 2 or 3), hydroxylamine (N(OH)_{z}(alkyl or aryl)_{2-z} where z is an integer possibly being 1 or 2), hydrazine, azo (N═N-alkyl or aryl)), diazonium, amide (CO—NH_{w}(alkyl or aryl)_{2-w} where w is an integer possibly being 0, 1 or 2) or cyano.

More specifically, the compounds of the invention are such that the three groups L₁, L₂ and L₃ together form a donor ligand of two electrons via a nitrogen atom, for example the groups L₁, L₂ and L₃ may together form terpyridine or 2-(2-pyridyl)-1,10-phenanthroline, optionally substituted on one or more carbon atoms of its pyridine rings by at least one halogen atom, a radical alkyl, aryl, hydroxyl, alcoxyl (O-alkyl), aryloxyl (O-aryl), carboxylic acid, ester (CO₂-alkyl), thiol, thioether (S-alkyl), sulfinic acid, sulfonic acid, nitro, nitroxyl, amine (N(alkyl or aryl)ₓH₂₋ₓ where x is an integer possibly being 0, 1 or 2), trialkylammonium (N(alkyl or aryl)_{y}H_{3-y}⁺ where y is an integer possibly being 0, 1, 2 or 3), hydroxylamine (N(OH)_{z}(alkyl or aryl)_{2-z} where z is an integer possibly being 1 or 2), hydrazine, azo (N═N-(alkyl or aryl)), diazonium, amide (CO—NH_{w}(alkyl or aryl)_{2-w} where w is an integer possibly being 0, 1 or 2) or cyano. Preferably, the substituent is an aryl radical, preferably phenyl, advantageously itself substituted by a methyl or methoxyl radical.

According to the invention, the terms <<bipyridine>>, <<phenanthroline>>, <<terpyridine>> and <<2-(2-pyridyl)-1,10-phenanthroline>> are such as defined below, respectively corresponding to the ligands 5, 6, 7 and 8:

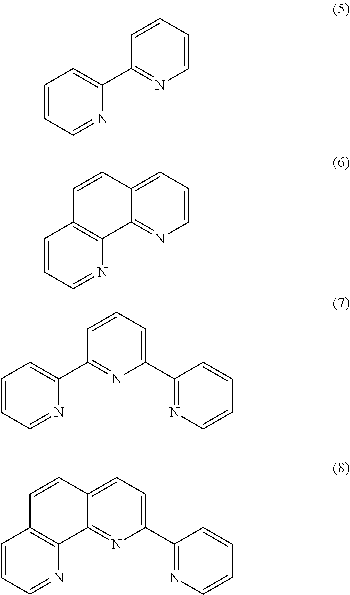

Y⁻ in the compounds of the invention is a counter-ion and is only present in the compound when the ruthenium complex carries a positive charge. Y⁻ is preferably a scarcely nucleophilic anion such as BF₄⁻, B(C₆H₅)₄⁻, PF₆⁻, CF₃SO₃⁻, tosylate (p-tolyl SO₃⁻), mesylate (MeSO₃⁻), SO₄²⁻, CF₃CO²⁻, CH₃CO₂⁻, bicarbonate (HCO₃⁻), ClO₄⁻ or NO₃⁻, in particular PF₆⁻.

The ligands L₁, L₂ and L₃ are so-called ancillary ligands whose presence is only necessary to complete the coordination sphere of ruthenium. It would seem that their nature has no influence of consequence on the biological activity of the claimed compounds, such activity being chiefly related to the type of the metallocycle(s).

According to one particular embodiment of the invention, m equals 1.

The curved line, with X₁, X₂ and Ru in the general formula, represents a ring. This ring is generally formed of 5 to 8 atoms (including the atoms represented by X₁, X₂ and Ru in formula (I)) preferably 5 to 6 atoms and further preferably 5 atoms. Typically the atoms of the said ring (other than those represented in the general formula) are selected from among carbon, nitrogen, oxygen and sulfur atoms. Preferably, these atoms are only carbon atoms. Each of these atoms may independently of the said ring form straight or cyclic structures, whether or not saturated, for which there are no particular limitations.

The curved line, with X₁ N and Ru in the general formula, represents another ring whose Ru—X₁ bond is common with the first ring. This ring is generally formed of 5 to 8 atoms [including the atoms represented by X₁, N and Ru in formula (I)], preferably 5 to 6 atoms and further preferably 5 atoms. Typically, the atoms of the said ring (other than those represented in the general formula) are selected from among carbon, nitrogen, oxygen and sulfur atoms. Preferably, these atoms are only carbon atoms. Each of these atoms may, independently of the said ring, form straight or cyclic structures whether or not saturated, for which there are no particular limitations.

Therefore, among the structural units including the two curved lines, particular mention may be made of the two groups shown below:

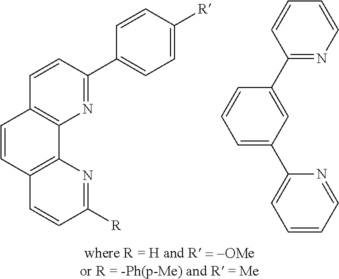

The compounds of the invention also include the optical and geometric isomers, taken individually or in a mixture (in particular the racemates), of the compounds of formula (I).

Among the compounds included in general formula (I) particular mention may be made of the compounds illustrated in FIG. 1 (compounds numbered (1) to (12)). According to one particular aspect, the compound of the invention comprises at least one of the compounds (1) to (12).

### Methods of Preparation:

Several methods exist for the synthesis of the compounds of the invention. The most advantageous is the method which uses the so-called intramolecular CH activation reaction or cyclometallation reaction (cf. A. D Ryabov, Chem. Rev. 1990, 90, 403-424) which leads directly to the formation of a C—Ru bond from a C—H bond.

Another method comprises the prior metallation of the carbon intended to be linked to ruthenium, for example by causing the corresponding ligand to react with a strong base such as n-BuLi, which leads to an organolithium whose lithium is then substituted by ruthenium. This second synthesis pathway was successfully used to obtain compounds similar to compound (1) but for which the pyridine groups of the ligand bis(2-pyridyl)-1,3-benzene were replaced by a CH₂NMe₂ entity (see Organometallics, 1996, 15, 941-948).

Compound (1) was obtained using the first pathway as per the following equation:

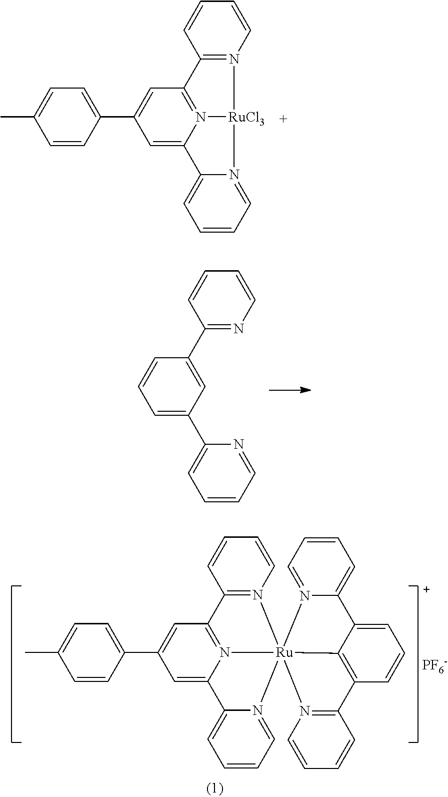

following the protocol described in the following publications: J. Am. Chem. Soc. 1991, 113, 8521-8522 and Inorg. Chem. 1993, 32, 4539-4543.

The compounds (2) and (3) were also synthesized using the first pathway following the protocol described in Eur. J. Inorg. Chem. 2000, 113-119. The reaction schemes are given below:

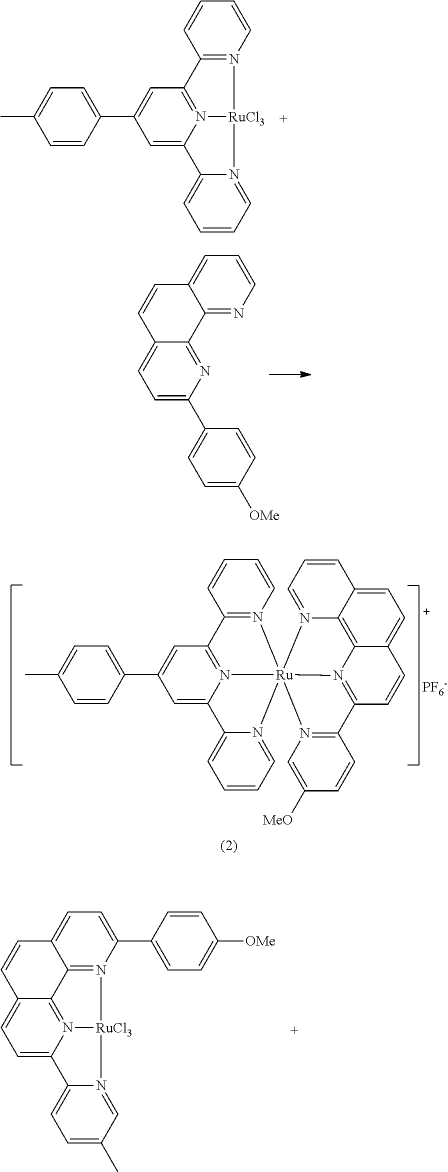

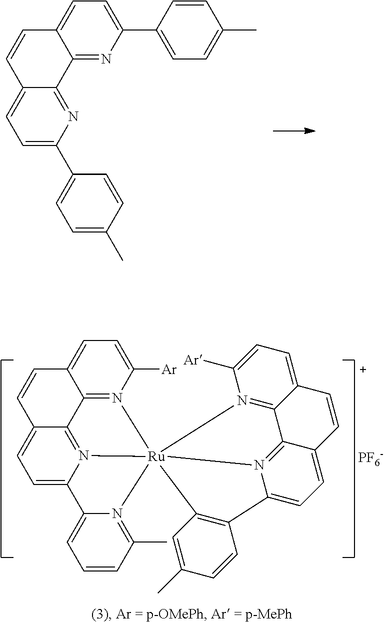

The compounds (4) to (7) and (10) to (12) were also synthesized using a similar synthesis pathway, described in Inorg. Chem, 2009, 5685-5696.

Compounds (8) and (9) were synthesized by metallation of the ligands 2,6-bis(2′pyridyl)-4-carbomethoxybenzene and 2,6-bis(2′pyridyl)-4-methylbenzene respectively.

The invention also concerns a ruthenium compound characterized in that it is selected from among the compounds (8) and (9):

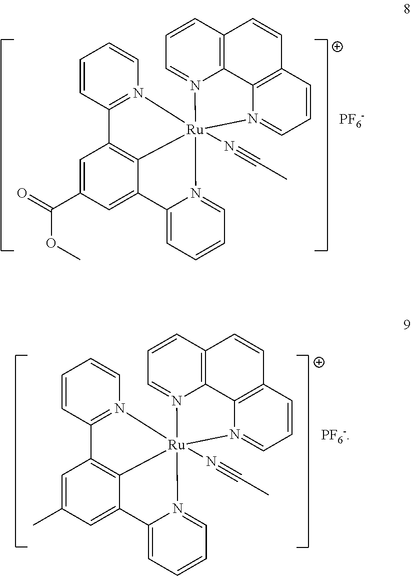

As specified above, the compositions of the invention are of particular advantage for treating diseases related to cell hyper-proliferation, cancers in particular. Cancers include those with solid or liquid tumours. Cancers relate in particular to glioblastomas, neuroblastomas, promyelocytic leukaemia, cancers of the prostate, ovaries, lungs, breast and digestive tract in particular the liver, pancreas, of the head and neck, colon, non-Hodgkin's lymphomas and melanomas.

A further object of the present invention is a compound of formula (I) such as defined above, for use in the treatment of diseases related to cell hyper-proliferation, cancers in particular.

A further object of the invention is the use of at least one compound of formula (I) such as defined above for the preparation of a pharmaceutical composition intended to treat diseases related to cell hyper-proliferation, cancers in particular.

The compounds of the present invention have an anti-proliferative effect on tumour cells. They are particularly useful for treating cancers by accumulation of the tumour cells in G0/G1 or G2/M phase, and optionally by inducing apoptosis or other type of cell death of tumour cells.

Without wishing to be bound by any theory of the invention, the compounds of the invention particularly appear to be capable of accumulating the tumour cells in phase G0/G1 or G2/M and therefore of blocking their cell cycle, but they also appear to be capable of generating their rapid death, in particular when their concentration is increased, a sign of dose-dependent toxicity.

In addition, the compounds of the invention are of particular advantage for treating tumours resistant to cisplatin or other anti-cancer drugs.

The composition of the invention can be administered in different manners and in different forms. For example, they can be administered systemically, via oral route, via inhalation or injection e.g. via intravenous, intra-muscular, sub-cutaneous, transdermal, intra-arterial route etc., the intravenous, intramuscular, subcutaneous, oral and inhalation routes being preferred. For injection, the compositions are generally in the form of liquid suspensions which can be injected using syringes or infusions for example. In this respect, the compounds are generally dissolved in saline, physiological, isotonic, buffered solutions, etc. compatible with pharmaceutical use and known to persons skilled in the art. Therefore the compositions of the invention may contain one or more agents or vehicles selected from among dispersants, solubilizers, stabilizers, preserving agents etc. Agents and vehicles which can be used in liquid and/or injectable formulations are in particular methylcellulose, hydroxymethylcellulose, carboxymethylcellulose, polysorbate 80, mannitol, gelatin, lactose, vegetable oils, acacia, etc.

The compositions may also be administered in the form of gels, oils, tablets, suppositories, powders, soft capsules, hard capsules, aerosols etc., optionally by means of galenic forms or devices ensuring sustained and/or delayed release. For this type of formulation, advantageously an agent is used such as cellulose, carbonates or starches.

Evidently, the rate of injection and/or the injected dose can be adapted by those skilled in the art in relation to the patient, to the pathology concerned, to the method of administration, etc. Typically, the compounds of the invention are administered at doses possibly varying between 0.1 μg and 100 mg/kg body weight, more generally between 0.01 and 10 mg/kg, typically between 0.1 and 10 mg/kg. In addition, repeat injections can be given. Also, for chronic treatments, systems with sustained and/or delayed release may be advantageous.

The invention also concerns a method for treating a pathology related to cell hyper-proliferation, in particular a cancer, via the administration of a composition according to the invention to an individual suffering from said pathology.

Among the other types of cell hyper-proliferation whose treatment can be envisaged according to the invention, mention may be made of benign tumours.

Within the context of the invention, the term <<treatment>> denotes preventive, curative, palliative treatment and the management of patients (reduced suffering, improved lifetime, slowed advance of the disease, reduced tumour growth, etc). The treatment may also be given in combination with other chemical or physical agents or treatments (chemotherapy, radiotherapy, gene therapy, etc.). The treatments and medicinal products of the invention are more particularly intended for humans.

Therefore, the compounds of the invention can advantageously be used in combination with an anti-cancer treatment using radiation such as radiotherapy.

According to another aspect of the invention, the compositions of the invention can be used with other chemical agents or anti-cancer therapeutic treatments such as the following therapeutic chemical agents: cisplatin, carboplatin, taxoter or taxol, advantageously taxol. The compounds of the invention are preferably packaged and administered in combined, separate or sequential manner relative to other therapeutic agents or treatments.

The present invention also concerns a method for the in vivo, in vitro or ex vivo inhibition of the proliferation of tumour cells, comprising the contacting of said tumour cells with a composition according to the invention. The tumour cells may in particular be those of the above-specified pathologies.

### KEY TO THE FIGURES

In the following figures and examples, the term <<RDC>> denotes a compound derived from ruthenium (Ruthenium Derived Compound).

FIG. 1: Examples of formula (I) compounds.

FIG. 2: Results of MTT test for the A172 cells of compounds 1, 2 and 3. The graphs are a mean of 8 points with standard deviations in a representative experiment out of 4 conducted experiments. The thick horizontal line indicates the IC₅₀ for each graph.

FIG. 3: Results of the MTT test for the A172 cells of compounds 4 to 12: percentage cell viability in relation to the concentration (μM) of the compound concerned. The thick horizontal line indicates the IC₅₀ for each graph.

FIG. 4: Profile of the cell cycle of HCT116 cells treated with compounds 8 and 9 at different concentrations.

FIG. 5: Expression of the CHOP protein and phosphorylation of histone H2AX on the serine site 137 for A172 tumour cells treated with compound 8 or compound 9, and comparison with the cells treated with the reference compound.

FIG. 6: Graph showing the tumour volume (in mm³) in relation to the number of treatment days with compound 9, in mice with F10B16 tumour.

FIG. 7: Graph showing the tumour volume (in mm³) in relation to the number of treatment days with compound 8, in mice with F10B16 tumour.

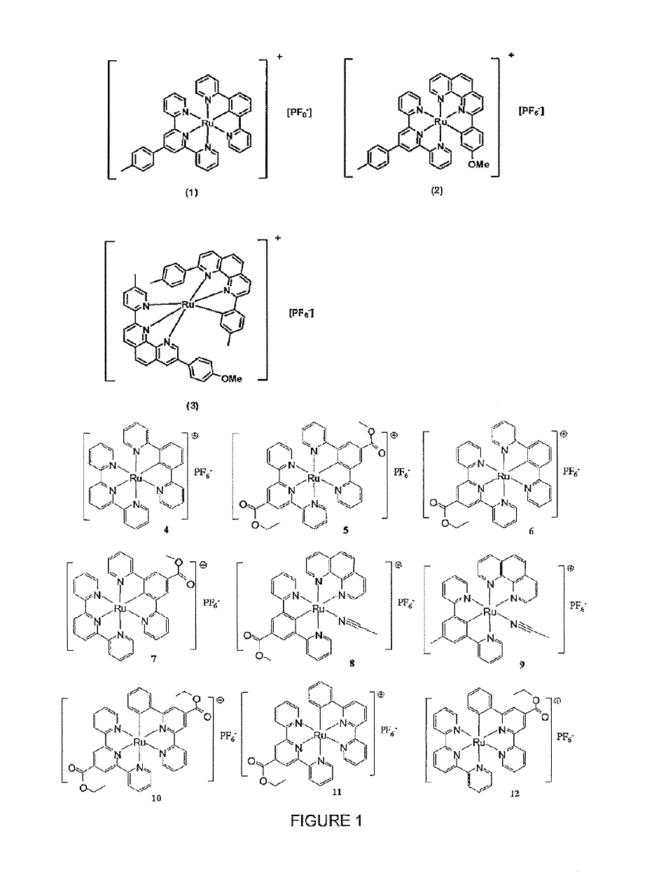

Other aspects and advantages of the present application will become apparent on reading the following examples which are to be considered as illustrative and non-limiting.

### EXAMPLES

### Example 1

### Synthesis and Characterization of Compounds 8 and 9

Cyclometallated compounds are sensitive to oxygen in air and to acids, especially during the metallation phase of the ligand by ruthenium. It is therefore advisable to conduct operations in a controlled atmosphere (nitrogen or argon) using the Schlenk tube technique. The solvents are dried and distilled under nitrogen (or argon) before use thereof.

### Synthesis of Compound 8

To a Schllenk tube are added 2,6-bis(2′pyridyl)-4-carbomethoxybenzene (L¹H) (62 mg, 0.2 mmol), [RuCl₂(η (eta)⁶-C₆H₆)]₂ (52 mg, 0.1 mmol), KPF₆(80 mg, 0.4 mmol), NaOH in flakes (8 mg, 0.2 mmol) in 4 mL of MeCN. This mixture is heated to 45° C. for 15 h. The orange solution obtained is filtered under an inert atmosphere on a standardized alumina column (column length: 15 cm in a tube of diameter 2 cm) and this solution is concentrated to reduce its volume to 0.5 mL. The addition of 10 mL Et₂O produces the precipitation of an orange product which meets the empirical formula [RuL¹(MeCN)₃]⁺PF₆⁻ (yield 91 mg, 69%).

A solution of [RuL¹(MeCN)₃]⁺PF₆⁻ (18 mg, 0.027 mmol) and of 1,10-phenanthroline (5.4 mg, 0.027 mmol) in MeOH (2 mL) is heated under reflux for 12 h. The solvent is then removed in vacuo, a dark brown powder is obtained. This powder is dissolved in 5 mL of MeCN and filtered through standardized alumina (column length: 5 cm in a tube of diameter 2 cm) using MeCN as eluent. The purple fraction is collected and the solvent evaporated in vacuo. The product 8 is obtained in the form of dark purple crystals from a solution of the preceding powder in a minimum volume of CH₂Cl₂ in which pentane is caused to diffuse slowly (yield: 18 mg, 88%).

MS (ES, m/z): Calculated for C₃₂H₂₄F₆O₂Ru: 612.098 (M). Found: 612.109.

IR (cm⁻¹): 2264 (vN≡C), 840 (vPF).

¹H NMR (400.13 MHz, CD₃CN, 300K): 10.02 (dd, 1H, ³J=5.1, ⁴J=1.8, H₀), 8.81 (dd, 1H, ³J=8.3, ⁴J=1.3, Hₚ), 8.62 (s, 2H, H₅), 8.35 (dd, 1H, ³J=8.1, ⁴J=4.9, Hₘ), 8.21 (d, 1H, ³J=8.8, Hₚₕₑₙ), 8.20 (d, 2H, ³J=8.0, H₁), 8.06 (dd, ³J=8.1, ⁴J=1.2, H₀), 7.99 (d, 1H, ³J=8.9, Hₚₕₑₙ), 7.70 (td, 2H, ³J=7.6, ⁴J=1.7, H₂), 7.57 (d, 2H, ³J=7.57, H₃), 7.32 (dd, 1H, ³J=5.5, ⁴J=1.2, Hₚ), 7.11 (dd, 1H, ³J=8.1, ⁴J=4.9, Hₘ), 6.81 (td, 1H, ³J=6.6, ⁴J=1.3, H₄), 4.05 (s, 3H, CH₃), 2.10 (s, 3H, NCCH₃).

¹³C NMR (100.62 MHz, CD₃CN, 300K): 169.2, 168.5, 155.3, 153.8, 151.2, 146.3, 145.4, 137.1, 136.1, 134.8, 131.7, 131.1, 128.7, 128.4, 127.0, 125.2, 124.4, 123.4, 120.7.

Anal: Calculated C₃₂H₂₄F₆N₅O₂PRu: C, 50.80; H, 3.20; N, 9.26. Found: C, 50.02; H, 3.25; N, 9.26%.

### Synthesis of Compound 9

The synthesis method was identical to the one used to obtain 8, with the difference that the starting ligand this time was 2,6-bis(2′pyridyl)-4-methylbenzene (L²H). Compound 9 was obtained in the form of dark purple crystals with a similar yield to the yield observed for 8.

MS (ES, m/z): Calculated: C₃₁H₂₄F₆Ru: 568.108 (M). Found: 568.119.

IR (cm⁻¹): 2265 (medium, vN≡C), 841 (strong, vPF).

¹H NMR (300 MHz, CD₃CN, 300K): 10.01 (dd, 1H, ³J=5.0, ⁴J=1.3, H₀), 8.76 (dd, 1H, ³J=8.1, ⁴J=1, Hₚ), 8.31 (dd, 1H, ³J=8.1, ⁴J=5.0, Hₚₕₑₙ), 7.99-8.04 (m, 3H, H₀+, H₁+Hₚₕₑₙ), 7.97 (s, 2H, H₅), 7.65 (td, 2H, ³J=7.7, ⁴J=1.5, H₂), 7.52 (m, 2H, H₃), 7.37 (td, 1H, ³J=5.5, ⁴J=1.3, Hₚ), 7.14 (dd, 2H, ³J=5.4, ⁴J=2.7, Hₘ), 6.73 (td, 2H, ³J=6.6, 4J=1.3, H₄), 2.66 (s, 3H, CH₃), 2.09 (s, 3H, NCCH₃).

¹³C NMR (100.62 MHz, CD₃CN, 300K): 165.5, 155.4, 153.8, 151.1, 145.0, 136.8, 135.4, 134.0, 131.7, 131.1, 130.2, 128.7, 128.3, 126.9, 125.6, 124.9, 122.6, 120.1,

Anal.: Calculated for C₃₁H₂₄F₆N₅PRu:

C, 52.25; H, 3.39; N, 9.83. Found: C, 50.87; H, 3.21; N, 9.70%.

### Example 2

### Analysis of the Cytostatic and Cytotoxic Effects of Ruthenium Derived Compounds on Cultures of Human Cell Lines of Glioblastoma and Cancer of the Colon

The first step in the characterization of the anti-cancer effects of the ruthenium derived compounds comprises testing their activity on tumour lines held in culture, and comparing these effects on lines which have different characteristics of resistance to anti-cancer treatments. A human glioblastoma line (A172) and a colon cancer line (HCT116) were used to test the cytostatic effects of the compounds according to the invention. Cisplatin was chosen as cytotoxic agent for comparison. As first approach, a MTT test was used to measure the activity of a mitochondrial enzyme which gives an estimation of the number of cells.

### Results

Several ruthenium derived compounds reduced the number of tumour cells on one or more of the lines tested, A172, HCT116. For the most active compounds (compounds (1) to (12)) this effect was observed at a concentration similar to or less than that of cisplatin. On the basis of these experiments, IC₅₀ values were estimated corresponding to the concentration needed to reduce by half the quantity of tumour cells present compared with the control condition. These results are summarized in Table 1.

TABLE 1

| IC50 μM | A-172 | HCT-116 |
|---|---|---|
| Compound (1) | 0.1-1 | 0.1-1 |
| Compound (3) | 0.1-1 | 0.1-1 |
| Compound (2) | 1-5 | 1-5 |
| Compound (4) | 0.5-0.7 | |
| Compound (5) | 1.4-1.6 | |
| Compound (6) | 0.4-0.8 | |
| Compound (7) | 1.0-1.4 | |
| Compound (8) | 1.5-1.9 | |
| Compound (9) | 0.6-1.0 | |
| Compound (10) | 0.8-1.0 | |
| Compound (11) | 0.9-1.1 | |
| Compound (12) | 1.0-1.4 | |

### Methods: MTT Test

The experiments were performed under a vertical laminar flow hood. The cell growth medium was composed of DMEM (Dulbecco's Modified Eagle's Medium), HEPES, 10% FCS (Foetal Calf Serum), 5% PS (Penicillin, Streptomycin) and was stored at +4° C., and PBS (Phosphate Buffer Saline, pH=7.4). Trypsin-EDTA (0.25% trypsin in 1 mM Na₄(EDTA)) and FCS were stored at −15° C. and unfrozen before use. MTT (4,5-dimethylthiazol-2-diphenyltetrazolium bromide) is a yellow solid produced by Aldrich. It was placed in sterile aqueous solution at a concentration of 5 mg/ml and stored at +4° C. It was diluted to 10% in the cell culture medium when used as staining agent of the living cells.

The human colon cancer cells (HCT-116) or glial cells (A-172) were purchased from European Type Culture Collection and placed in an incubator at 37° C., 5% CO₂ in round Petri dishes (diameter 10 cm) with 10 mL of medium. When they were sufficiently numerous (70% confluence) they were washed in PBS at ambient temperature then mixed with 1.5 mL of Trypsin-EDTA to detach them from the Petri dish. This cell suspension was placed in culture medium heated to 37° C., and then this solution was spread on 96-well cell culture plates (100 μL/well) which were left to incubate for 24 to 48 hours, until a cell confluence of 50% was reached. The medium was renewed with cell medium containing different concentrations of RDCs and cisplatin at 37° C., which were left to incubate. After 48 hours, the medium was replaced by a MTT solution at 37° in the medium, which was placed in the incubator for at least one hour or until violet crystals derived from the complexing of MTT were formed quantitatively at the bottom of each well. Finally, this medium was replaced by 100 μL/well of a 0.04M solution of HCl/¹PrOH at ambient temperature to dissolve the crystals. The optical density of the solutions obtained was read off. The optical densities of the wells treated with RDCs or cisplatin were compared with those of non-treated wells (controls).

A manipulation comprised the treating of 4 plates (3×RDC and cisplatin). Each plate contained a single product at different concentrations. 9 columns were treated at 50, 20, 15, 10, 7.5, 5, 2.5, 1 and 0.2 μM and 3 columns were left as controls. The control column located at each end of the plate was not taken into account. The third control column was taken into account for calculations.

Determination of IC₅₀ and statistical Newmann-Keuls variance tests were performed using Prism GraphPad software v.4.

### Results of the MTT Test:

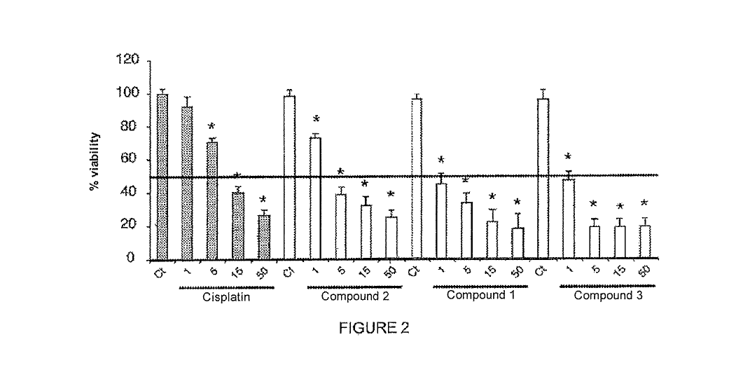

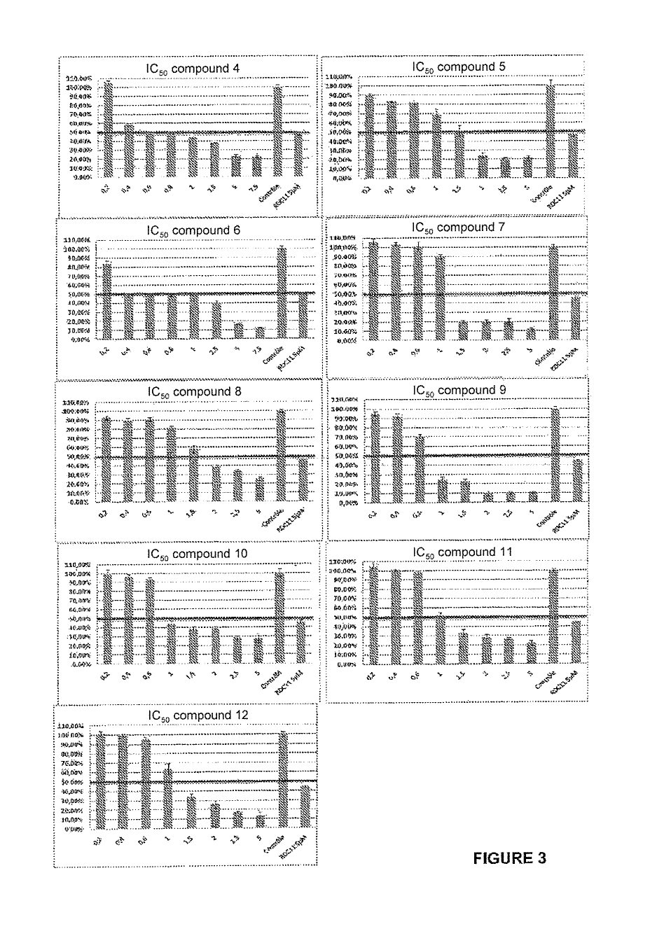

The results of the MTT tests are given in FIGS. 2 and 3 for the A172 cells.

The A172 wells were cultured in 96-well plates in DMEM medium with 10% calf serum. At 50% confluence, the cells were treated for 48 h with cisplatin (cisp) or the various ruthenium derived compounds at the indicated concentrations (1, 5, 15, 50 μM). The quantity of cells present in the wells was evaluated using a MTT test (MTT, Sigma) whose reaction products were quantified with an Elisa plate reader (Metertech, USA) (490-650 nm). The results obtained were compared with the values of the control condition (100% viability).

### Example 3

### Study of the Inhibition of the Proliferation of Cancer Cells by the Compounds of the Invention

The capability of compounds 8 and 9 to inhibit the proliferation of cancer cells was studied using the previously described MTT method, which gives an estimation of the number of cells. These studies were conducted on cancer lines of various origins (see Table 2) under the previously described culture conditions. The IC₅₀ values (μM) were determined for each of the lines and compared with the values obtained for a reference ruthenium derived compound. In all the experiments performed, the two compounds of the invention were more efficient than the reference compound described in WO 2006/016069 and meeting the following formula:

TABLE 2

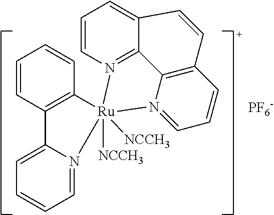

| Cancer | | IC₅₀ | IC₅₀ | IC₅₀ |
|---|---|---|---|---|
| line | Origin | Compound 8 | Compound 9 | Reference |
| HCT116 | Colon cancer | 1-2 | 0.1-1 | 2-5 |
| A2780 | Ovarian cancer | 1-2 | 0.1-1 | 2-5 |
| 3LL | Lung cancer | 1-2 | 0.1-1 | 2-5 |
| U87 | Glioblastoma | 1-2.5 | 1-2 | 2-7.5 |
| N2A | Neuroblastoma | 1-2 | 0.1-1 | 2-5 |
| Fadu | Head & neck cancer | 1-2 | 0.1-1 | 2-5 |
| Katto III | Pancreatic cancer | 1-2 | 0.1-1 | 2-5 |

### Example 4

### Study of the Profile of the Cell Cycle Subsequent to Treatment with the Compounds of the Invention

The effect of compounds 8 and 9 on the cell profile was studied using the FACS technique (Fluorescence Activated Cell Sorter i.e. the association of a flow cytometer and a cell sorter) after staining the cells with propidium iodide to visualize the quantity of cell DNA. These analyses allow visualization of the cell cycle phase at which the cells accumulate subsequent to treatment with the compounds of the invention, and possibly whether cell death by apoptosis has been induced.

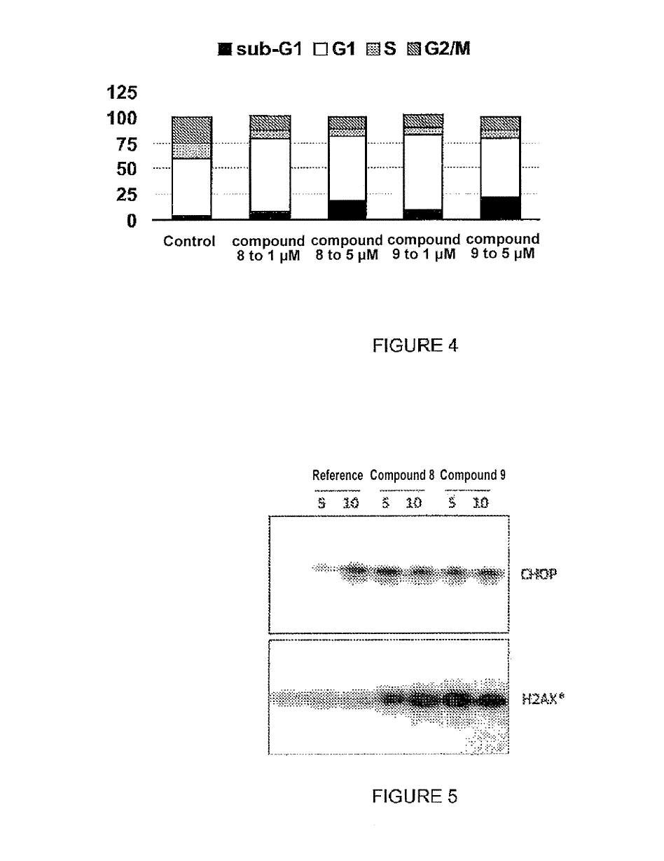

The results shows that in the HCT116 line, the compounds 8 and 9 induce a halt in the cell cycle at phase G1 or cell death (characterized by the sub-G1 phase) in relation to the concentration of drug used (see FIG. 4).

In this experiment, the HCT116 cells were treated for 48 h at the indicated doses (1 μM and 5 μM). After a treatment time of 48 h, the cells were washed three times with PBS (phosphate buffer saline) and fixed in 90% ethanol. The cells were then stained with a solution of propidium iodide/RNase. The stained cells (10 000 per sample) were then counted using a FACScan flow cytometer and CellQuest software (Becton Dickinson) to determine the DNA content and the profile of the cell cycle.

### Example 5

### Study of the Acute Toxicity of Compounds According to the Invention

The acute toxicity induced by compounds 8 and 9 was studied in Black-6 mice aged 8 weeks after injection of single doses of product at different concentrations (2, 7.5, 15, 30 and 60 μmol per kg). The doses inducing 50% mortality (LD₅₀) were calculated and are given in Table 3 below. As comparison, the acute toxicity of the above-described reference compound is given. This experiment showed that compound 8 has greater toxicity (22.5 μmol/Kg) than the reference compound (57.5 μmol/Kg), whereas the toxicity of compound 9 was lower (>60 μmol/Kg).

In this experiment, the injections were given via intraperitoneal route with groups of 5 animals per dose. The weight and survival of the animals were followed up over 15 days.

TABLE 3

| Product | LD₅₀ (μmol/Kg) |
|---|---|
| Compound 8 | 22.5 |
| Compound 9 | >60 |
| Reference | 57.5 |

### Example 6

### Study of the Mechanisms of Action of the Compounds According to the Invention

To determine via which mechanisms of action compounds 8 and 9 could lead to slowing of cell proliferation or to cell death, the activation of two specific markers, the expression of the CHOP protein and phosphorylation of the histone H2AX on the serine site 137 were monitored. The CHOP protein has pro-apoptotic properties (induction of cell death). Phosphorylation of the H2AX histone is a marker of DNA damage which leads to activation of p53, a protein having pro-apoptotic properties or the property of slowing cell proliferation.

The results show that the compounds 8 and 9 are more efficient in inducing expression of the CHOP protein and phosphorylation of the H2AX histone than the reference compound (see FIG. 5).

In these experiments, the tumour cells A172 were treated for 6 hours with the compounds at two concentrations (5 μM and 10 μM). After the 6 hour treatment time, the cells were lysed and 30 g of soluble proteins were separated on 12% polyacrylamide gel. After migration, the proteins were transferred onto nitrocellulose using the Western blot technique. The proteins CHOP and H2AX were then detected using specific primary antibodies, themselves recognized by corresponding secondary antibodies whose peroxidase activity was identified using an ECL kit.

### Example 7

### Study of the Anti-Tumour Activity of Compounds According to the Invention

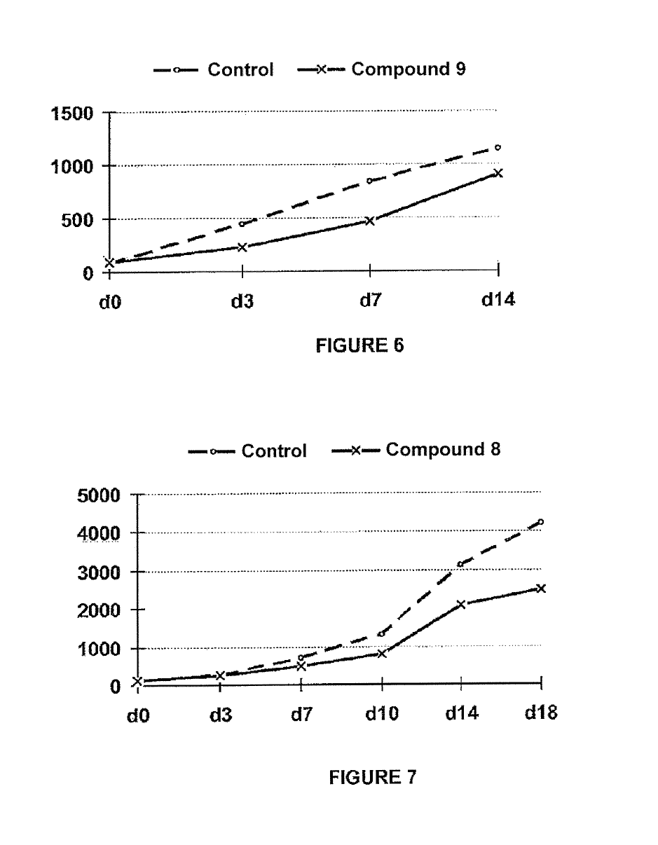

To determine the anti-cancer potential of these molecules, we analyzed the effects of compound 8 and compound 9 on tumour growth, using the in vivo melanoma model F10B16 implanted in Black-6 mice. These effects were compared with those of the reference compound. The chronic treatments (twice a week at 7.5 μmol/kg) in mice having tumours led to a reduction in tumour growth. This reduction in tumour growth (treated tumour volume/non-treated tumour volume: T/C) was of the order of 80% for compound 8 and 60% for compound 9 (see Table 4 and FIGS. 6 and 7).

| Products | T/C (%) |
|---|---|
| Compound 8 | 81% |
| Compound 9 | 58% |
| Reference | 61% |

In these experiments, the F1PB16 cells (200 000 cells) were implanted sub-cutaneously in Black-6 mice aged 8 weeks. Groups of 6 mice per condition were formed. The treatments were started when the tumours were palpable with a volume of the order of 100 mm³.

1. A pharmaceutical composition which, in a pharmaceutically acceptable medium, comprises at least one complex ruthenium compound of the following general formula: 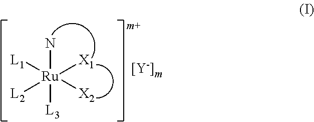
wherein:
L₁, L₂, L₃, are the same or different and represent either a donor ligand of 2 electrons via a nitrogen, oxygen, phosphorus or sulphur atom, or a halogen atom,
Y⁻ is a counter-ion (when m=1),
m is 0 or 1,
X₁ and X₂ differ from each other, one representing a nitrogen atom and the other a carbon atom,
between X₁ and X₂, represented by a first curved line, there is a succession of atoms which, together with X₁, X₂ and Ru represented in the formula, final a ring composed of 5 to 8 atoms, and
between N and X₁, represented by a second curved line, there is a succession of atoms which, together with the nitrogen atom, X₁ and Ru represented in the formula, foam a ring composed of 5 to 8 atoms.

2. The composition according to claim 1, characterized in that L₁, L₂ and/or L₃ represent a pyridine, bipyridine, phenanthroline or terpyridine group.

3. The composition according to claim 1 characterized in that the three groups L₁, L₂ and L₃ together form a donor ligand of two electrons via a nitrogen atom.

4. (canceled)

5. The composition according to claim 1 characterized in that Y⁻ is selected from the group consisting of BF₄⁻, B(C₆H₅)₄⁻, PF₆⁻, CF₃SO₃⁻, tosylate (p-tolylSO₃⁻), mesylate (MeSO₃⁻), SO₄²⁻, CF₃CO₂⁻, CH₃CO₂⁻, bicarbonate (HCO₃⁻), ClO₄— and NO₃⁻.

6. The composition according to claim 1, characterized in that m equals 1.

7. The composition according to claim 1, characterized in that one or both of the first curved line and the second curved line represents a ring formed of 5 to 6 atoms.

8. The composition according to claim 7, characterized in that the atoms of the ring or rings are all carbon atoms.

9. The composition according to claim 1, characterized in that the complex ruthenium compound comprises one of the following structural units:

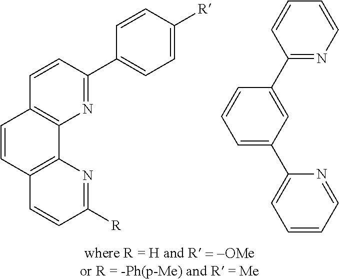

10. The composition according to claim 1, characterized in that the complex ruthenium compound is selected from the group consisting of:

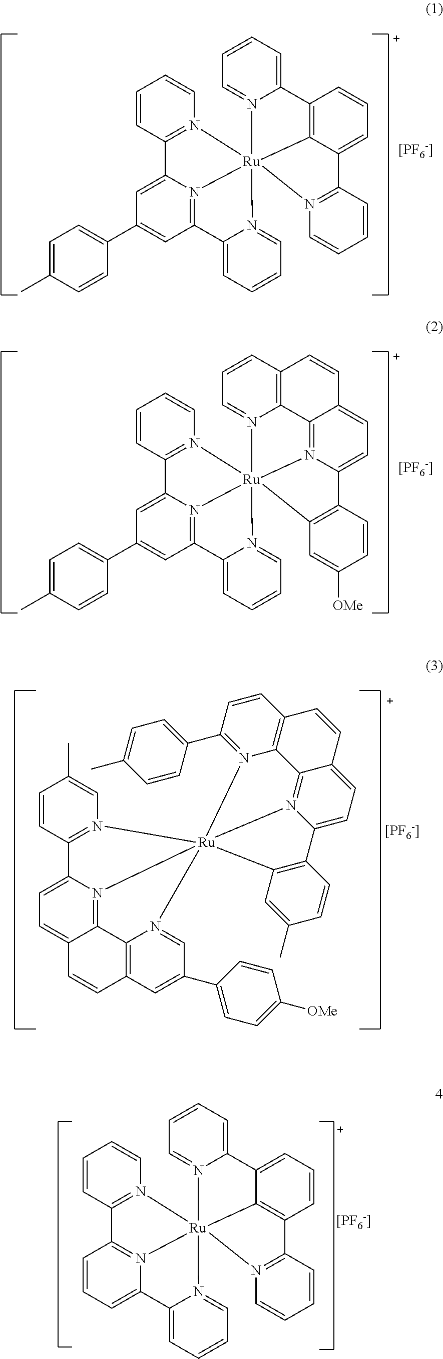

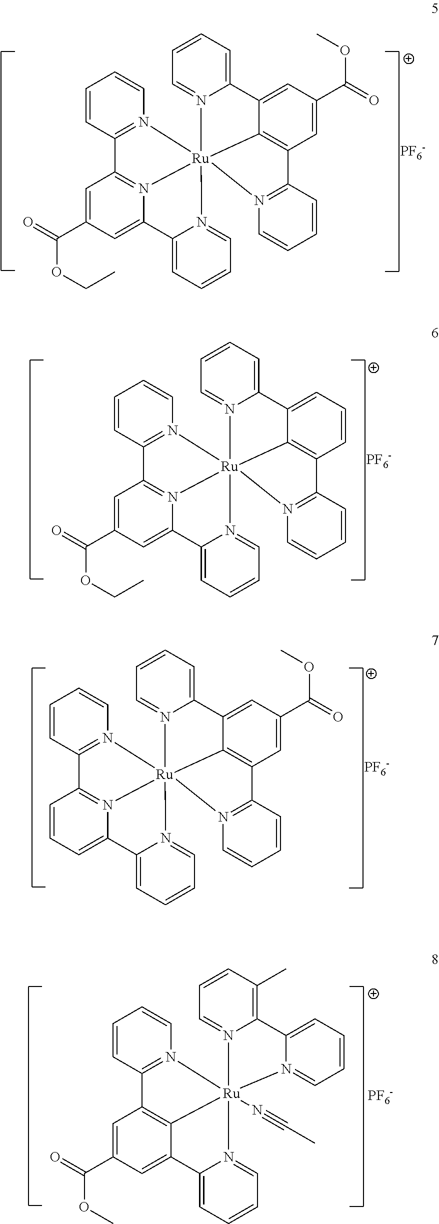

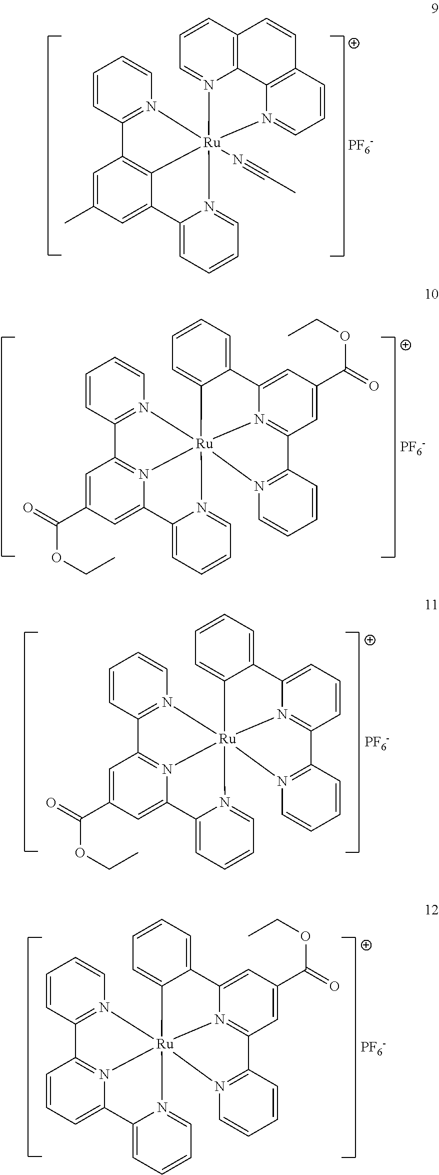

11. The composition according to claim 10, characterized in that the compound is the complex of formula (I).

12-18. (canceled)

19. A ruthenium compound, characterized in that it is selected from among the compounds (8) and (9):

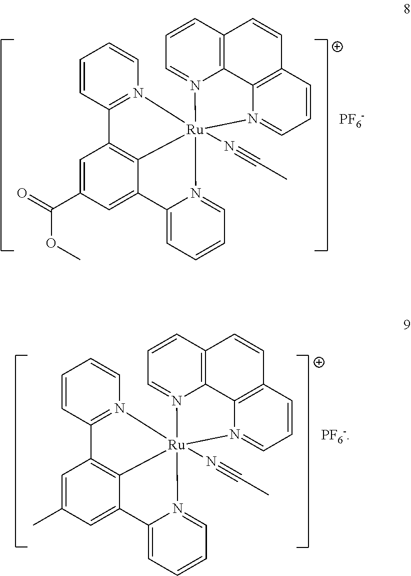

20. The composition according to claim 2, wherein said pyridine, bipyridine, phenanthroline or terpyridine group is substituted by at least one substituent selected from the group consisting of: halogen atom, an alkyl radical, an aryl radical, hydroxyl, alcoxyl (O-alkyl), aryloxyl (O-aryl), carboxylic acid, ester (CO₂-alkyl), thiol, thioether (S-alkyl), sulfinic acid, sulfonic acid, nitro, nitroxyl, amine (N(alkyl or aryl)ₓH₂₋ₓ where x is 0, 1 or 2), trialkylammonium (N(alkyl or aryl)_{y}H_{3-y}⁺ where y is 0, 1, 2 or 3), hydroxylamine (N(OH)_{z}(alkyl or aryl)_{2-z} where z is 1 or 2), hydrazine, azo (N═N-(alkyl or aryl)), diazonium, amide (CO—NH_{w}(alkyl or aryl)_{2-w} where w is 0, 1 or 2), and cyano

21. The composition according to claim 3, wherein said donor ligand of two electrons is selected from the group consisting of terpyridine, 2-(2-pyridyl)-1,10-phenanthroline, substituted terpyridine and substituted 2-(2-pyridyl)-1,10-phenanthroline.

22. The composition according to claim 21, wherein said substituted terpyridine and said substituted 2-(2-pyridyl)-1,10-phenanthroline are substituted, on one or more carbon atoms of a pyridine ring, by at least one substituent selected from the group consisting of: a halogen atom, an alkyl radical, an aryl radical, hydroxyl, alcoxyl (O-alkyl), aryloxyl (O-aryl), carboxylic acid, ester (CO₂-alkyl), thiol, thioether (S-alkyl), sulfinic acid, sulfonic acid, nitro, nitroxyl, amine (N(alkyl or aryl)ₓH₂₋ₓ where x is 0, 1 or 2), trialkylammonium (N(alkyl or aryl)_{y}H_{3-y}⁺ where y is 0, 1, 2 or 3), hydroxylamine (N(OH_{z}(alkyl or aryl)_{2-z} where z is 1 or 2), hydrazine, azo (N═N-(alkyl or aryl)), diazonium, amide (CO—NH_{w}(alkyl or aryl)_{2-w} where w is 0, 1 or 2) and cyano.

23. The composition according to claim 22, wherein said aryl radical is a phenyl substituted by methyl or methoxyl.

24. A method of treating a disease related to cell hyper-proliferation in a patient in need thereof, comprising the step of
administering to the patient, in a pharmaceutically acceptable medium, a therapeutically effective amount of at least one complex ruthenium compound of the following general formula: 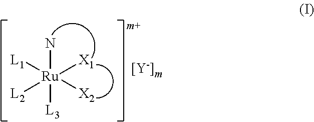
in which:
L₁, L₂, and L₃, are the same or different and represent either a donor ligand of 2 electrons via a nitrogen, oxygen, phosphorus or sulphur atom, or a halogen atom,
Y⁻ is a counter-ion (when m=1),
m is 0 or 1,
X₁ and X₂ differ from each other, one representing a nitrogen atom and the other a carbon atom,
between X₁ and X₂, represented by a first curved line, there is a succession of atoms which, together with X₁, X₂ and Ru represented in the formula, form a ring composed of 5 to 8 atoms, and
between N and X₁, represented by a second curved line, there is a succession of atoms which, together with the nitrogen atom, X₁ and Ru represented in the formula, form a ring composed of 5 to 8 atoms.

25. The method of claim 24, wherein the disease related to cell hyper-proliferation is cancer.

26. The method of claim 25, wherein said cancer is selected from the group consisting of glioblastomas, promyelocytic leukaemia, prostate cancer, ovarian cancer, lung cancer, breast cancer, digestive tract cancer, liver cancer, pancreatic cancer, head and neck cancer, colon cancer, non-Hodgkin's lymphoma, and melanoma.

27. The method of claim 25, wherein said method is used to treat tumours resistant to cisplatin or to other anti-cancer drugs.

28. The method of claim 25, wherein the method is carried out in combination with anti-cancer radiation therapy.

29. The method of claim 25, wherein said at least one complex ruthenium compound is administered in combination with at least one other anti-cancer chemical agent.

30. The method of claim 24, wherein said at least one complex ruthenium compound and said at least one other anti-cancer chemical agent are: packaged in combination and administered in combination, packaged separately and administered in combination, or packaged separately and administered in a sequential manner.

31. The method of claim 24, wherein said at least one complex ruthenium compound treats said cancer by causing accumulation of tumour cells in phase G0/G1 or G2/M phase, and optionally by induction of apoptosis or another type of cell death.
